(19) United States
(12) Plant Patent Application Publication
(10) Pub. No.: US 2004/0237158 P1
(43) Pub. Date: Nov. 25, 2004

(54) Grape plant named 'Frontenac gris'

(76) Inventors: James Luby, St. Paul, MN (US); Peter Hemstad, Edina, MN (US)

Correspondence Address: BIOLOGICAL PATENT SERVICES, LLC, 14015 42ND AVE N., PLYMOUTH, MN, 55446-3827, US

(21) Appl. No.: 10/775,435

(22) Filed: Feb. 10, 2004

Related U.S. Application Data
(60) Provisional application No. 60/446,660, filed on Feb. 11, 2003.

(51) Int. Cl. A01H 5/00; A01D 34/52; A01D 34/73

(52) U.S. Cl. PLT/205

(57) ABSTRACT

The invention is a new and distinct variety of grape plant designated ‘Frontenac gris’, which produces gray colored fruit suitable for white wine production, and has a combination of high wine quality, excellent cold hardiness and disease resistance, and very good productivity.

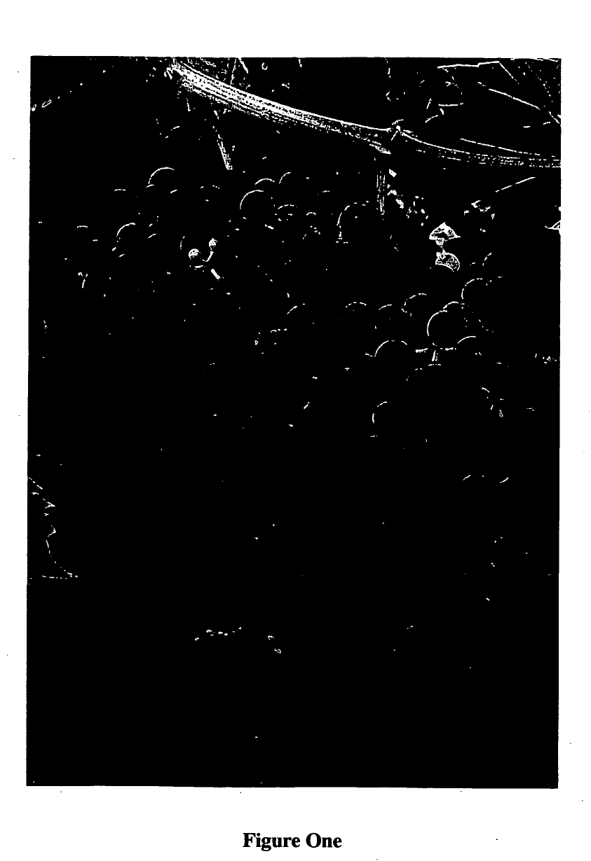

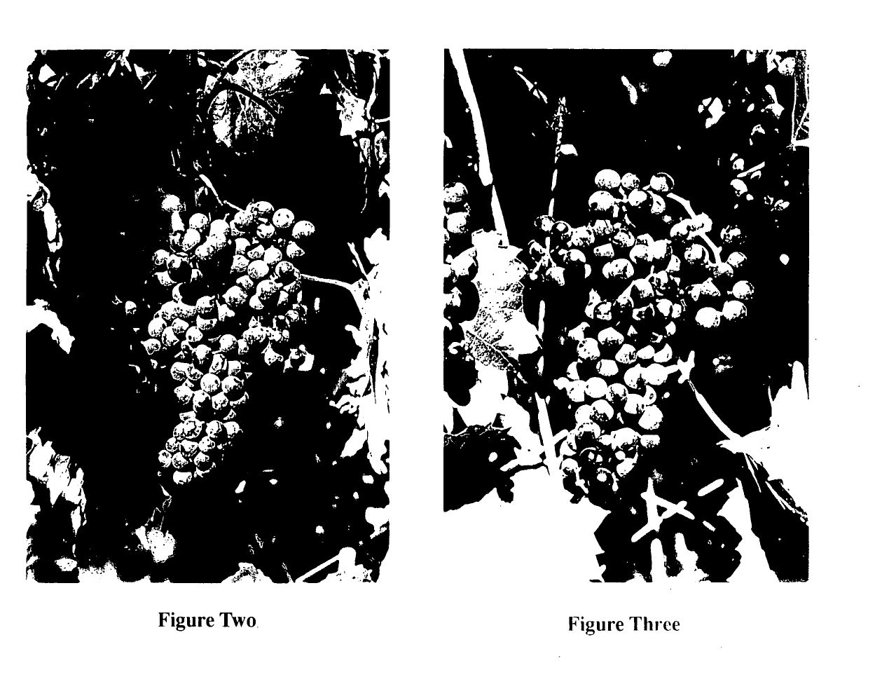

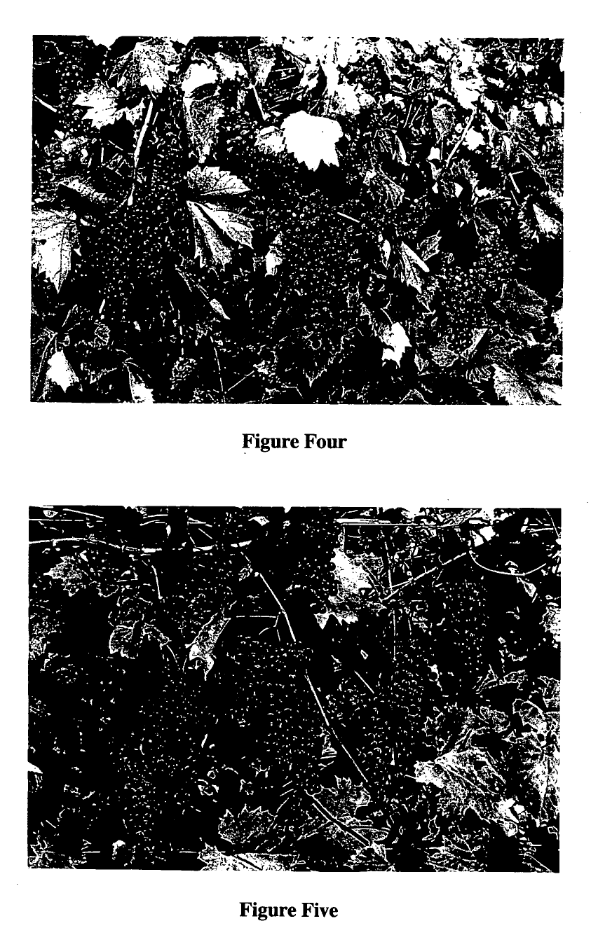

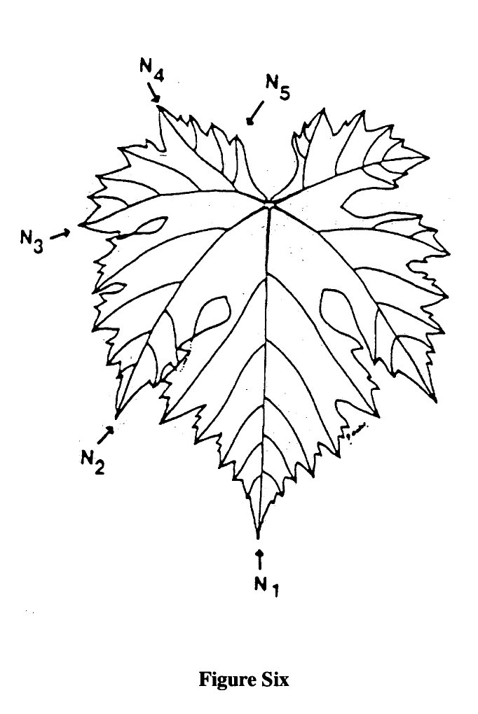

### REFERENCE TO A PROVISIONAL APPLICATION

This application claims the benefit of U.S. Provisional Application No. 60/44660, filed Feb. 02, 2003 under U.S.C. 119(e).

### BOTANICAL CLASSIFICATION

Vitis hybrid

### VARIETY DENOMINATION

‘Frontenac gris’

### BACKGROUND OF THE INVENTION

Most grape varieties used for production of high quality wines around the world are of the species Vitis vinifera. These V. vinifera varieties, when cultivated in northern regions of the United States with a continental climate, are often subject to serious injury or death from low temperatures during winter. Although several wild Vitis species occur in colder regions of North America and eastern Asia, the wine made from these species generally has serious defects. Thus, there is a need for grape varieties that are winter hardy, yet produce fruit capable of yielding high quality wine. A grape breeding program at the University of Minnesota has been engaged in developing such varieties since the early 1980s.

### BRIEF SUMMARY OF THE INVENTION

‘Frontenac gris’ is a variety of grape (Vitis hybrid) with gray (or gris, in French) colored fruit suitable for white wine production, and is well adapted to the Upper Midwest climate of the United States. ‘Frontenac gris’ has an unusual combination of high wine quality, excellent cold hardiness and disease resistance, and very good productivity. ‘Frontenac gris’ was originally identified as a sport of ‘Frontenac’ (unpatented), a variety of grape of hybrid origin with bluish black colored fruit with red juice_suitable for red wine production that was introduced in 1996 by the grape breeding program at the University of Minnesota Horticultural Research Center (HRC) in Carver County, Minnesota.

‘Frontenac gris’ propagates readily from hardwood cuttings, with young vines quick to become established, and all ‘Frontenac gris’ plants propagated in this manner have been genetically stable, producing only gray colored fruit with clear juice. As grown in east central Minnesota, the plants of ‘Frontenac gris’ are vigorous, productive, and winter hardy. The vines of ‘Frontenac gris’ have relatively few tendrils and an open growth habit well suited to upper cordon training systems. The budbreak and bloom of ‘Frontenac gris’ are early to midseason, and its flowers are perfect and self-fertile. ‘Frontenac gris’ vines typically set a moderate to heavy crop. The fruit of ‘Frontenac gris’ is borne on medium sized clusters that are usually somewhat loose, and the berries are small and gray with a waxy bloom at maturity. Berry splitting and bunch rots have rarely been observed, even under wet conditions in the autumn harvest season. In some years, over-cropping may occur and cluster thinning may be required. In east central Minnesota, the fruit typically ripens around September 29, about three days after fruit of the ‘Seyval’ variety (unpatented), and at harvest is usually relatively high in both sugar and acidity. When grown in Minnesota, the fruit of ‘Frontenac gris’ has a high titratable acidity which usually requires either malolactic fermentation or residual sugar in order to produce a well balanced wine, and when grown in regions experiencing greater degree day accumulations, the acidity has been substantially lower.

The fruit of ‘Frontenac gris’ can be fermented to produce either white table wine or dessert wine, and such wines have been well received in various tastings. The wine tends to have good body and pleasant aromas, with very little of the herbaceous qualities associated with V. riparia and many interspecific grape hybrids. ‘Foxy aromas’ derived from V. labrusca have not been detected. The most common aroma component identified by tasters has been peach, but apricot, citrus, and tropical fruit aromas have also been noted. At times the wine may exhibit a slight pink or peach coloration derived from the lightly pigmented skin of the fruit.

‘Frontenac gris’, like ‘Frontenac’, has exhibited resistance to several important diseases in evaluations. Even under conditions of high disease pressure, ‘Frontenac gris’ is highly resistant to downy mildew (Plasmopara viticola) on both the foliage and the fruit. ‘Frontenac gris’ is moderately resistant to powdery mildew (Uncinula necator), which has been observed frequently at low levels on the foliage, but has not been seen on the fruit. ‘Frontenac gris’ is moderately resistant to black rot (Guignardia bidwellii), which has been observed sporadically and at low levels on the foliage and the fruit. Anthracnose (Elsinoe ampelina) has rarely been observed on the foliage and fruit of ‘Frontenac gris’. ‘Frontenac gris’ is susceptible to the foliar form of grape phylloxera (Daktulosphaira vitifoliae) while tolerant to the root form of this disease. ‘Frontenac gris’ is tolerant to the adverse effects of phenoxy herbicide drift.

‘Frontenac gris’ has proven to be sufficiently cold hardy for consistent production in east central Minnesota where temperatures frequently reach −35° C. during the winter season. Field tests have shown ‘Frontenac gris’ to be at least as cold hardy as the ‘Foch’ variety (unpatented), and substantially cold hardier than the ‘Seyval’ variety and most other presently available grape cultivars used for wine production.

### BRIEF DESCRIPTION OF THE FIGURES

The accompanying color photographs were taken in mid September and represent typical mature berry clusters and vines of ‘Frontenac gris’ and ‘Frontenac’ as grown under standard field conditions in Excelsior, Minn. The Photograph in Figure One is a close-up view of clusters of berries of ‘Frontenac gris’. The photographs in Figure Two and Figure Three provide a comparison between the berry clusters of ‘Frontenac gris’ (Figure Two) and ‘Frontenac’ (Figure Three). Figure Four and Figure Five are photographs comparing mature vines of ‘Frontenac gris’ and ‘Frontenac’, respectively. Figure Six is a drawing taken from Dettweiler E., 1991, ‘Preliminary Minimal Descriptor List for Grapevine Varieties’, Institüt fur Rebenzüchtung, Geilweilerhof, Germany. The colors in the photographs are as close as possible with the photographic and printing technology utilized. The color values cited in the detailed botanical description accurately describe the colors of the new grape.

### DETAILED BOTANICAL DESCRIPTION

‘Frontenac gris’ arose from the spontaneous mutation of the ‘Frontenac’ variety in 1992. ‘Frontenac’ arose from a controlled cross as part of the grape breeding program at the University of Minnesota Horticultural Research Center (HRC) in Carver County, Minnesota, and originated from the cross designated GE 7828 and made in 1978 between the French hybrid variety ‘Landot 4511 ’ (unpatented) and the University of Minnesota Vitis riparia clone #89 found growing wild near Jordan, Minn. The ‘Frontenac’ parental variety was originally tested as MN 1047 and is described in The Brooks and Olmo Register of New Fruit and Nut Varieties, Third Edition, 1997, p. 265.

‘Frontenac gris’ originated as a single cane sport bearing gray colored fruit on a plant of the bluish black fruited ‘Frontenac’ variety growing at location Block 10 Row 16 Panel 7 at the HRC in 1992. When ripe, the berries of ‘Frontenac gris’ are gray and contain only slight amounts of anthocyanin pigment, whereas the ‘Frontenac’ parental variety produces highly pigmented dark bluish black berries. A total of 8 vines of ‘Frontenac gris’ were asexually propagated by hardwood cuttings from this original cane and planted at the HRC as follows: 2 plants were planted in 1995 in Block 10 Row 17 Panel 11; 3 plants were planted in 1996 in Block 18 Row 8 Panel 3; and 3 plants were planted in 1999 in Block 18 Row 11 Panel 5. These plants were observed through 2002, including their flowers and fruit, and were indistinguishable in appearance from the original ‘Frontenac gris’ cane. Therefore, the asexual progeny of ‘Frontenac gris’ are stable and reproduced true to type in successive generations.

The following data pertain to vines grown at the University of Minnesota Horticultural Research Center in Carver County, Minnesota near Excelsior. For comparison purposes, data were collected for certain morphological descriptors from fruit of the variety ‘Seyval’, a grape variety commonly grown in Minnesota and the eastern United States for the production of white wine. Alphanumeric color designations refer to values based on the 1995 R.H.S. Colour Chart published by The Royal Horticultural Society, London, England. Many of the descriptors are based on those set forth by the International Board for Plant Genetic Resources in collaboration with the Office Internationale de la Vigne et du Vin (OIV) and the International Union for the Protection of New Varieties of Plants.

When dimensions, sizes, colors and other characteristics are given, it is to be understood that such characteristics are approximations set forth as accurately as possible. Variations of the usual magnitude incident to climatic factors, fertilization, pruning, pest control and other cultural practices are to be expected.

A) Mature Canes

The values presented are the means (with ranges in parentheses) of 10 canes observed from the 2002 growing season.

| | | |
|---|---|---|
| 1. | Color of canes: | striated, reddish brown |
| | | RHS colors 166A, 166C |
| 2. | Internode length at base: | 2.4 cm (2.1-3.0) |
| 3. | Internode length at midpoint: | 9.3 cm (7.5-12.2) |
| 4. | Lenticels present: | yes |
| 5. | Cane cross-section shape: | elliptical |
| 6. | Density of hairs on mature cane: | none |
| 7. | Tendril pattern on shoot: | 2, 0, 2, 0 etc. (two nodes with |
| | | a tendril followed by one node |
| | | without) |
| 8. | Tendrils forked: | yes |
| 9. | Tendril texture: | striated |
| 10. | Tendril length: | 15 cm (11-20) |
| 11. | Bud width: | 3.8 mm (3.1-5.0) |
| 12. | Bud shape: | triangular |

B) Trunk

The observations presented are from the 2002 growing season.

| | |
|---|---|
| 1. Bark texture | somewhat flaky, small vertical |
| | segments approx. 1-2 cm. × 4-6 cm. |
| 2. Bark color | silver-gray |
| | RHS colors 201B, 201C |

C) Mature Leaves

Ten representative mature leaves from above the clusters in the middle third of the shoot were examined. The leaves were pressed and dried for later analysis. The values presented below are means (with ranges in parentheses) from collections in September 2000. Descriptors of mature leaves, including the designations N1 through N5, relate to “OIV—Code Numbers 065-093” of Preliminary Minimal Descriptor List for Grapevine Varieties (Dettweiler E., 1991, Institüt fur Rebenzüchtung, Geilweilerhof, Germany).

| | | ‘Frontenac gris’ |
|---|---|---|
| 1. | Length of blade: | 15.3 cm (13.3-18.3) |
| 2. | Width of blade: | 15.5 cm (12.5-18.2) |
| 3. | Shape of blade: | circular-kidney shaped |
| 4. | Number of lobes: | 4.4 (3-5) |
| 5. | Length of vein N1: | 12.6 cm (10.5-15.0) |
| 6. | Length of vein N2: | 11.5 cm (9.7-13.5) |
| 7. | Length of vein N3: | 8.5 cm (7.3-9.3) |
| 8. | Length of vein N5: | 4.2 cm (2.9-5.5) |
| 9. | Length of N2 teeth: | 13.3 mm (10-16) |
| 10. | Width of N2 teeth: | 14.0 mm (11-16) |
| 11. | Length/width ratio | 0.96 (0.71-1.1) |
| | of N2 teeth: | |
| 12. | Length of N4 teeth: | 8.2 mm (7-9) |
| 13. | Width of N4 teeth: | 11.9 mm (10-18) |
| 14. | Length/width ratio | 0.71 (0.5-0.9) |
| | of N4 teeth: | |
| 15. | Shape of teeth: | rectilinear-convex |
| 16. | Shape of petiolar sinus: | wide open |
| 17. | Shape of base | u-shaped |
| | of petiolar sinus: | |
| 18. | Depth of petiolar sinus: | 22.4 mm (17-29) |
| 19. | Width of petiolar sinus: | 48.6 mm (39-61) |
| 20. | Length of petioles: | 7.2 cm (4.7-9.6) |
| 21. | Shape of upper sinuses: | open |
| 22. | Shape of base of upper | u-shaped |
| | sinuses: | |
| 23. | Pubescence on | none |
| | adaxial surface: | |
| 24. | Pubescence on | very sparse on main veins |
| | abaxial surface: | and at petiolar junction |
| 25. | Color of adaxial | 146B, yellow-green |
| | leaf surface: | |
| 26. | Color of abaxial | 146C, yellow-green |
| | leaf surface: | |
| 27. | Color of leaf | 59C, red-purple |
| | Petiole | |

D) Young Shoots

The observations presented are from the 2002 growing season.

| | ‘Frontenac gris’ |
|---|---|
| 1. Form of shoot tip: | closed by small leaves |
| 2. Density of prostate hairs | none |
| on tip: | |
| 3. Density of erect hairs | very sparse |
| on tip: | |
| 4. Petiole pigmentation: | dark red on adaxial, |
| | light red on abaxial |
| 5. Shoot pigmentation: | adaxial striped to solid |
| | purple, abaxial striped |

E) Flowers

| | |
|---|---|
| 1. Fragrance: | moderately fragrant |
| 2. Mean time | June 14 when grown |
| of flowering: | in Excelsior, Minnesota |
| 3. Color of petal: | 145A, yellow-green |
| 4. Color of sepal: | 144A, yellow-green |
| 5. Color of pollen: | 4B, yellow |
| 6. Petal number: | 5, fused in calyptra |
| 7. Petal shape: | cohering at summit and separating |
| | at base: 2.5 mm long; 1 mm wide at |
| | fused end; reflexed after dehiscence |
| | from flower |
| 8. Shape of cluster: | somewhat conical, typically with one |
| | shoulder |
| 9. Size of cluster: | 14.5 cm long (range 10.1-19.8); |
| | 6.3 cm wide (range 3.6-11.1) |
| 10. Number of flowers | 190 (range 111-278) |
| per cluster: | |
| 11. Size of individual | 5.6 mm long; 4.1 mm wide |
| entire flower: | |
| 12. Pollen fertility: | yes, based on use in controlled |
| | pollinations |
| 13. Color of stamen: | Anther: 162C, grayed-yellow |
| | Filament: 155A, white |
| 14. Stamen number: | 4.9 (range 4-6) |
| 15. Pistil number: | 1 per flower |
| 16. Pistil length: | 2.5 mm |
| 17. Color of pistil: | 144A, yellow-green |

F) Fruit

The values presented below are means (with ranges in parentheses) from fruit observed in the 2000 growing season, except for those traits indicated (**), which are means from the 2000-2003 growing seasons for ‘Frontenac gris’ and from the 1995 and 1999-2002 growing seasons for ‘Seyval’.

| | ‘Frontenac gris’ | ‘Seyval’ |
|---|---|---|
| 1. Cluster length: | 17.4 cm (14.7-22.4) | 12.1 cm (9.0-15.1) |
| 2. Cluster weight:** | 137.1 g (88-193) | 162.4 g (92-298) |
| 3. Cluster density: | loose-medium | medium |
| 4. Berry weight:** | 1.13 g (1.02-1.09) | 1.90 g (1.59-2.22) |
| 5. Berry length: | 11.4 mm (10.2-12.1) | 13.8 mm (12.2-15.4) |
| 6. Berry diameter | 11.3 mm (10.1-12.2) | 13.3 mm (12.1-15.3) |
| at equator: | | |
| 7. Berry shape: | roundish | roundish |
| 8. Berry cross- | circular | circular |
| section: | | |
| 9. Berry, color of | gray-golden | yellow-green |
| skin: | | |
| | RHS colors 199B, 201B | RHS color 151A |
| 10. Berry, color of | light green | light green |
| flesh: | RHS colors 160A, 160B | RHS color 150D |
| 11. Berry, particular | lightly fruity | neutral |
| flavor: | (peach, kiwi) | |
| 12. Length of pedicel: | 5.7 mm | 6.2 mm |
| 13. Berry, separation | difficult | difficult |
| from pedicel: | | |
| 14. Berry, presence | fully developed | fully developed |
| of seeds: | | |
| 15. Seed number/ | 2.4 (2-4) | 2.2 (1-4) |
| berry: | | |
| 16. Seed length: | 0.53 mm (0.50-0.55) | 0.59 mm (0.54-0.65) |
| 17. Seed width: | 0.33 mm (0.31-0.36) | 0.39 mm (0.35-0.46) |
| 18. Seed length/ | 1.61 | 1.51 |
| width ratio: | | |
| 19. Seed weight: | 0.023 g | 0.031 g |
| 20. Seed color: | RHS color 165A | RHS color 177A |

G) Harvest Parameters

Values represent the means (with ranges in parentheses) for fruit harvested over four growing seasons (1999-2003) for ‘Frontenac gris’ and six growing seasons (1995, 1996, 1999-2002) for ‘Seyval’.

| | ‘Frontenac gris’ | ‘Seyval’ |
|---|---|---|
| 1. Harvest date: | 9/29 (9/18-10/5) | 9/26 (9/16-10/6) |
| 2. Brix: | 26.3° (24.6°-26.8°) | 20.9° (18.6°-23.2°) |
| 3. pH: | 3.06 (2.85-3.18) | 3.11 (2.91-3.41) |
| 4. % titratable acidity: | 1.22% (1.06-1.41%) | 0.86% (0.72-1.02%) |

H) Vineyard Performance

Based on observations compiled over four years (1999-2003).

| | |
|---|---|
| 1. Susceptibility to powdery mildew | moderate |
| (Uncinula necator): | |
| 2. Susceptibility to downy mildew | very low |
| (Plasmopara viticola): | |
| 3. Susceptibility to black rot (Guignardia bidwellii): | low-moderate |
| 4. Susceptibility to bunch rot (Botrytis, etc): | very low |
| 5. Susceptibility to foliar phylloxera | moderate- |
| (Daktulosphaira vitifoliae): | severe |
| 6. Susceptibility to crown gall | no natural |
| (Agrobacterium tumefaciens): | infection |
| | observed |
| 7. Susceptibility to phenoxy herbicide drift | low |
| (e.g., 2,4-D): | |
| 8. Berry splitting: | low |
| 9. Berry shelling: | low |
| 10. Vigor level: | high |
| 11. Winter hardiness: | high, trunks |
| | have survived |
| | −38° C. |
| 12. Wood ripening: | very good |

I) Wine Quality

Descriptions below are compiled from observations on wine made from ‘Frontenac gris’ fruit harvested during the 1999-2003 growing seasons.

| | |
|---|---|
| 1. Flavors and aromas: | peach, apricot, citrus, tropical fruit; |
| | no ‘hybrid’ , herbaceous, or labrusca aromas |
| 2. Balance: | good body, well balanced when finished |
| | with residual sugar or put through malolactic |
| | fermentation |
| 3. Color: | attractive light pink/peach unless filtered or |
| | fined |
| 4. Propensity for oxidation: | low |
| 5. Overall quality: | very good |

1. A new and distinct variety of grape plant designated ‘Frontenac gris’ as described and illustrated herein.
